(19) United States
(12) Patent Application Publication
(10) Pub. No.: US 2021/0270792 A1
(43) Pub. Date: Sep. 2, 2021

(54) A DETECTION SYSTEM FOR DETECTING MATTER AND DISTINGUISHING SPECIFIC MATTER FROM OTHER MATTER

(71) Applicant: Photonic Detection Systems Pty Ltd, Kingsley (Western Australia) (AU)

(72) Inventors: Kamal ALAMEH, Joondalup (Western Australia) (AU); Selam AHDEROM, Joondalup (Western Australia) (AU)

(21) Appl. No.: 17/260,530

(22) PCT Filed: Nov. 23, 2018
(86) PCT No.: PCT/AU2018/051251

§ 371 Date: Jan. 14, 2021

(30) Foreign Application Priority Data
Jul. 17, 2018 (AU) 2018902589

(51) Int. Cl. G01N 33/00; G01N 21/27; G01J 3/10; G01J 3/28; G06T 7/50

(52) U.S. Cl. CPC G01N 33/0098; G01N 21/27; G01J 3/10; G01N 2021/1765; G01J 3/2823; G06T 7/50; G01J 2003/2836; G01J 3/2803

(57) ABSTRACT

The present disclosure provides a detection system for detecting matter and distinguishing specific matter from other matter. The detection system comprises a spectral analysis system configured to at least assist in determining whether matter comprises the specific matter based on an intensity of light reflected from the specific matter. The spectral analysis system comprises a light source capable of emitting light having at least one known wavelength or wavelength range. Further, the spectral analysis system comprises an optical element for directing the emitted light towards the matter. The spectral analysis system also comprises a detector for detecting light reflected from the matter and a spatial analysis stem. The spatial analysis system is configured to at least assist in determining whether the matter comprises the specific matter based on a shape of the matter. The spatial analysis system comprising an image capturing device for capturing an image of the matter. Further, the spatial analysis system comprises an outcome determination system arranged to receive a first input from the spectral analysis system and a second input from the spatial analysis system, and determine an outcome providing an indication of whether the matter is specific matter based on the first and second inputs.

### TECHNICAL FIELD

The present disclosure relates to a detection system for detecting matter and distinguishing specific matter from other matter. The matter may be, but is not limited to, plant matter and the specific plant matter may include weeds.

### BACKGROUND

The control of weed growth is an important factor in agriculture. Large areas of plant matter including valuable plants, such as crops, and weeds are usually sprayed with expensive and toxic chemicals in order to control or restrain the weed growth. Ideally only the weeds should be sprayed, but this is difficult if the weeds grow amongst the valuable plant matter. It may also be useful to be able to distinguish in an automated manner particular plant matter from other matter so that the particular plant matter can be treated differently to the other matter

PCT International Application Number PCT/AU2007/001075, owned by the present applicant, discloses an optical device for discriminating specific plant matter from other matter. The optical device comprises laser diodes that emit light having three wavelengths and a plurality of light beams. Each light beam has the three wavelengths sequentially directed to the plant matter. A detector detects light beams that are reflected back from the plant matter. A processor then processes the reflected intensities and compares the detected intensity ratios at the three wavelengths with a library of such intensity ratios of known plant matter whereby the device is enabled to discriminate a particular type of plant matter from other matter.

WO 2011/143686 A1, also owned by the present applicant, discloses an automated device that is able to distinguish weeds from the valuable plant matter in a quick manner to restrict the spraying of the chemicals to the weeds only.

The present disclosure provides a further technological improvement.

### SUMMARY OF INVENTION

The present invention provides a detection system for detecting matter and distinguishing specific matter from other matter, the detection system comprising:
a spectral analysis system configured to at least assist in determining whether matter comprises the specific matter based on an intensity of light reflected from the specific matter, the spectral analysis system comprising:
   a light source capable of emitting light having at least one known wavelength or wavelength range;
   an optical element for directing the emitted light towards the matter; and
   a detector for detecting light reflected from the matter;
a spatial analysis system configured to at least assist in determining whether the matter comprises the specific matter based on a shape of the matter, the spatial analysis system comprising an image capturing device for capturing an image of the matter; and
an outcome determination system arranged to receive a first input from the spectral analysis system and a second input from the spatial analysis system, and determine an outcome providing an indication of whether the matter is specific matter based on the first and second inputs.

The spectral analysis system may produce a first output, the first output providing an indication of whether the matter is specific matter, wherein the first input is representative of the first output. Further, the spatial analysis system may produce a second output, the second output providing an indication of whether the matter is specific matter, wherein the second input is representative of the second output.

The outcome determination system may be arranged to determine whether the matter is specific matter if one of the following conditions is satisfied:
(i) at least one of the first input and the second input indicates that the matter is specific matter; and
(ii) each of the first input and the second input indicate that the matter is specific matter.

The outcome determination system may further comprise a user operable selector capable of allowing a user to select whether the outcome depends on the condition (i) or (ii).

The first input may be, or may be representative of, an output signal from the detector of the spectral analysis system.

The emitted light may have at least three wavelengths and the light source is configured to generate a combined beam of light having the at least three wavelengths.

The optical element may be configured to receive the combined beam of light and direct a plurality of component light beams towards the matter.

The optical element may have first surface portions through which the plurality of component light beams is capable of being directed to the matter including the specific matter, the first surface portions having optical properties that are selected so that light intensity differences between the component light beams are reduced.

The spatial characteristic may be a geometric shape of the specific matter.

The image capturing device may be a camera.

The matter may be plant matter and the specific matter may be unwanted plant matter.

The detection system may further comprise a dispenser for dispensing a substance to the unwanted plant matter, wherein the dispenser is arranged to selectively dispense the substance onto the specific matter when the outcome determination system determines that the plant matter is unwanted plant matter.

The spatial analysis system and the spectral analysis system may perform respective analyses substantially simultaneously and/or in real-time.

The outcome determination system may be configured to determine whether the matter is specific matter using an artificial neural network.

### BRIEF DESCRIPTION OF DRAWINGS

FIG. 1 is a block diagram of a detection system in accordance with an embodiment of the present invention;

FIG. 2 is a schematic illustration of a detection system in accordance with an embodiment of the present invention;

FIG. 3 is a block diagram of an outcome determiner in accordance with an embodiment of the present invention;

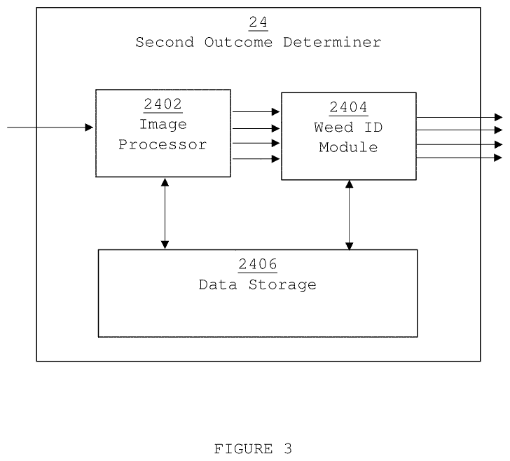

FIG. 4 is a schematic illustration of a neural network, which may be used in an embodiment of the detection system; and

FIG. 5 shows a plurality of images of matter for user according to an embodiment of the present invention.

### DETAILED DESCRIPTION

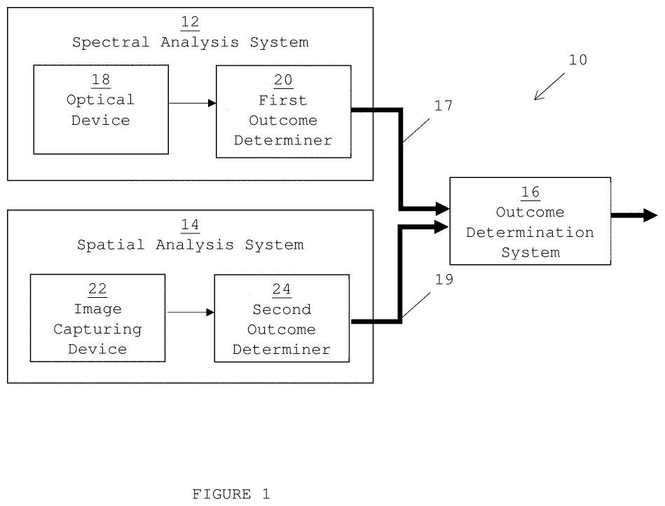

Referring to FIG. 1, there is shown an embodiment of a detection system 10 for detecting matter and distinguishing specific matter from other matter. In this example, the matter is plant matter, and the specific matter is weed. However, it will be appreciated that the matter and specific matter can be other types of matter.

The system 10 includes components that are related to a device disclosed in WO 2011/143686 A1, which is incorporated herein by reference.

The system 10 comprises a spectral analysis system 12, a spatial analysis system 14, and an outcome determination system 16.

The spectral analysis system 12 is configured to assist in determining whether plant matter is weed based on an intensity of light reflected from the plant matter. To this end, the spectral analysis system 12 comprises an optical device 18 for detecting intensities of light reflected from the plant matter. The optical device 18 is similar to the optical device disclosed in WO 2011/143686 A1, and therefore will only be described briefly below.

In this example, the spectral analysis system 12 further comprises a first outcome determiner 20, which receives and analyses an output signal from the optical device 18, and determines based on the intensities detected by the optical device 18 whether the plant matter is weed. The first outcome determiner 20 may comprise or form part of a computing device comprising a processor arranged to analyse information provided by the optical device 18.

The spatial analysis system 14 is also configured to assist in determining whether plant matter is weed, however does so based on a shape of the matter. To this end, the spatial analysis system 14 comprises an image capturing device 22 for capturing an image of the matter, and thus an image of the shape of the matter. The spatial analysis system 14 in this example also comprises a second outcome determiner 24 for analysing information indicative of the shape of the matter and determining whether based on the shape the matter is weed. The second outcome determiner 24 may comprise or form part of a computing device comprising a processor arranged to analyse information provided by the image capturing device.

The system 10 further comprises an outcome determination system 16 arranged to receive at least one first input 17 from the spectral analysis system 12 and at least one second input 19 from the spatial analysis system 14. It will be appreciated that although FIG. 1 represents the first and second inputs 17, 19 as a single line, there may be multiple first inputs 17 and second inputs 19. The outcome determination system 16 then determines an outcome based on the first and second input(s), which provides an indication of whether the plant matter is weed.

In this example, the outcome determinations system 16 receives a plurality of first inputs 17 and a plurality of second inputs 19. The first inputs 17 correspond to outputs produced by the first outcome determiner 20, and the second inputs 19 correspond to outputs produced by the second outcome determiner 24. Hereinafter, the inputs 17, 19 to the outcome determination system 16 may therefore interchangeably be referred to as outputs 17, 19 of the determines 20, 24, respectively. The outcome determination system 16 may also comprise or form part of a computing device comprising a processor arranged to analyse information provided by the first and second outcome determiners 20, 24.

Based on the result of the outcome determination system 16, the system 10 can identify in substantially real-time whether plant matter in an area of interest 1815 is to be classified as specific matter, such as weed. Consequently, the system 10 can also take an appropriate action if specific matter is identified. The action may for example be spraying a weed killer on the weed.

By utilising two types of information—spectral and shape-based information—in determining whether plant matter is weed, the system 10 may provide the advantage of a back-up form of identification if one of the types of information fails to adequately identify a weed. For example, if a type of weed has the same spectral characteristics as other plant matter but a distinctive shape, the spectral analysis system 12 may not identify it as a weed, but the spatial analysis system 14 may. Conversely, if a type of weed has the same shape as other plant matter but distinctive spectral characteristics, the spectral analysis system 12 may identify it as a weed, but the spatial analysis system 14 may not.

Additionally, it is possible for a user to define the conditions that need to be satisfied for the outcome determination system 16 to positively identify a weed. For example, since the outcome produced by the system 16 is based on two independent types of information, a user may select a setting that causes the system 16 to positively identify a weed if at least one of the spectral analysis system 12 and the spatial analysis system 14 has identified the matter as a weed. Alternatively, the user may select a setting that causes the system 16 to positively identify a weed only if both the spectral analysis system 12 and the spatial analysis system 14 have identified the matter as a weed. The conditions upon which appropriate action is to be taken can thus be defined and adjusted by the user.

Specific components of the system 10 will now be described in more detail.

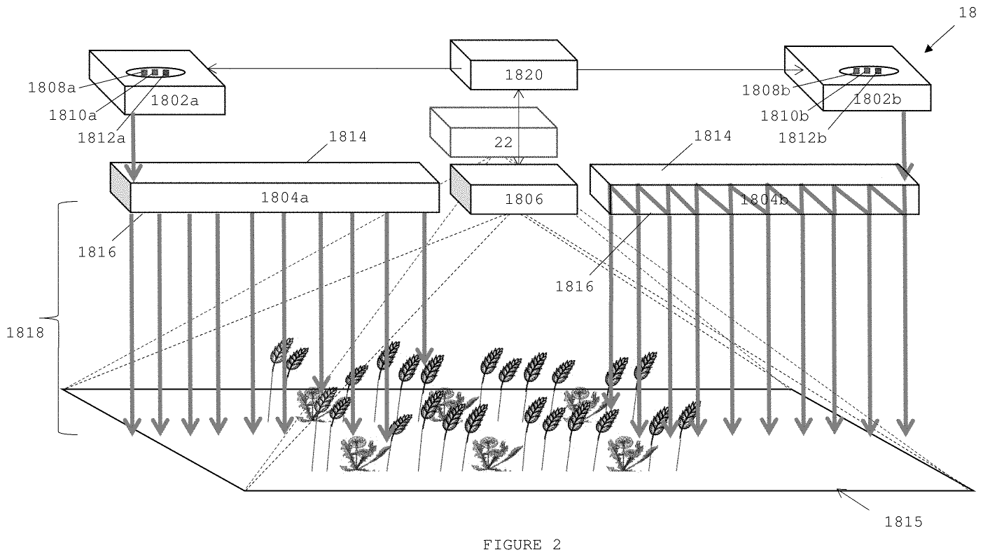

With reference to FIG. 2, the optical device 18 in this example comprises a first light source 1802a and a second light source 1802b, a first optical element 1804a and a second optical element 1804b, a detector 1806. The optical device 18 further comprises a local controller 1820 that controls and coordinates various functions of the optical device 18. The local controller 1820 may for example be a programmable microcontroller specifically programmed to carry out functions of particular functions of the optical device 18.

Each light source 1802a, 1802b is capable of emitting light having at least one known wavelength or wavelength range, and is associated with a respective optical element 1804a, 1804b. In particular, the first optical element 1804a is arranged to direct the light emitted from the first light source 1802a towards the matter in the area of interest 1815. Similarly, the second optical element 1804b is arranged to direct the light emitted from the second light source 1802b towards the matter.

In this example, the first and second light sources 1802a and 1802b are considered as a pair of light sources. Each light source 1802a, 1802b in the pair comprises three laser diodes each capable of generating light at different wavelengths. In particular, the first light source 1802a includes a first laser diode 1808a generating light having a first wavelength of 635 nm, a second laser diode 1810a generating light having a second wavelength of 670 nm and a third laser diode 1812a generating light having a third wavelength of 785 nm. Similarly, the second light source 1802b includes a first laser diode 1808b generating light having a first wavelength of 635 nm, a second laser diode 1810b, generating light having a second wavelength of 670 nm and a third laser diode 1812b generating light having a third wavelength of 785 nm.

The laser diodes 1808a, 1810a, 1812a from the first light source 1802a emit pulses of laser light in sequence. The pulses may be for any suitable length of time, such as but not limited to 200 microseconds. The laser pulses from each diode 1808a, 1810a, 1812a are directed by a beam combiner (not shown) in the same direction towards the optical element 1804a, such that the sequence of laser pulses form a single stream or light beam. The laser diodes 1808b, 1810b, 1812b from the second light source 1802b are arranged in the same manner. Furthermore, pairs of corresponding lasers 1808a/b, 1810a/b, 1812a/b one from each light source 1802a, 1802b which emit light having the same wavelength, are operated together and in sequence with other pairs of corresponding lasers.

Each optical element 1804a, 1804b is implemented as an optical cavity. The optical cavities each have opposite reflective coatings 1814 and 1816. The reflective coatings 1814, 1816 have a relatively high reflectivity, such as 99% or higher. Light from respective light sources 1802a, 1802b is transmitted toward the optical elements 1804a, 1804b and reflected between the reflective coatings 1814, 1816 in a zigzag manner (illustrated particularly in the optical element 1804b, but not shown for the element 804a, in FIG. 2). However, the reflective coatings 1816 on a lower surface of the optical elements 1804a, 1804b have lower reflectivities than the reflective coatings 1814 on an upper surface of the optical elements 1804a, 1804b. Thus, a portion of light is transmitted through the reflective coatings 1816 and a series of component light beams 1818 is formed and directed in a substantially parallel manner towards plant matter.

The detector 1806 is arranged to detect an intensity of the component light beams 1818 reflected from the plant matter in the area of interest 1815. In this regard, it is known that different types of plant matter, such as crops and weeds, can be distinguished on the basis of intensities of light reflected from them. As described briefly above in relation to PCT/AU2007/001075, a detector (such as the detector 1806) detects light beams that are reflected from the plant matter. A processor then processes the reflected intensities and compares the detected intensity ratios at the three wavelengths with a library of such intensity ratios of known plant matter. Weed can thus be distinguished from other plant matter if the corresponding detected intensity ratios match that of a weed in the library.

Regarding the detection of intensity ratios, as light is emitted by the light sources 1802a, 1802b, a portion of the component light beams 1818 is reflected by the plant matter and detected by the detector 1806. The detector 1806 comprises an imaging photodiode array, an objective lens and a filter (not shown). An output signal generated by the detector 1806 is representative of the intensities of the component light beams 1818 reflected from the plant matter. The detector 1806 communicates the output signal to the local controller 1820. The local controller 1820 then communicates information indicative of the signal as a series of inputs to the first outcome determiner 20 of the spectral analysis system, for processing an analysis. Each input in the series of inputs may for example correspond to one of detected intensities of one of the component light beams 1818.

As described above, the pairs of corresponding lasers 1808a/b, 1810a/b, 1812a/b are operated in sequence with other pairs of corresponding lasers at a predetermined operation period, such as 200 microseconds. Therefore, it is possible for the spectral analysis system to correlate a detected intensity with a respective wavelength so that wavelength specific intensity information is obtained by the detector 1806 and communicated to the controller 1820.

In particular, an objective lens of the detector 1806 is arranged to capture an image of the component beams 1818 reflected by the plant matter onto the photodiode array. The objective lens is arranged so that each component light beam 1818 is reflected at a position approximately 60 cm (±20 cm) below the device 18 are imaged onto respective cells of the photodiode array. Consequently, it is possible to detect intensities arising from respective reflections on the plant matter. Moreover, due to the known geometry of the optical device 18 and the parallel nature of the component light beams 1818, it is also possible for the spectral analysis system 12 to determine locations of plant matter, including any specific plant matter, at which the light was reflected. The system 12 is then capable of correlating the intensity information with the relevant location information.

In this example, the first outcome determiner 20 receives (via the local controller 1820) an output signal from the optical device 18. The first outcome determiner 20 then analyses the signal to correlate portions of the signal to respective locations of the component light beams 1818. For each component light beam 1818, the determiner 20 calculates the intensity ratio and compares the ratio to a library of ratios. Based on the comparison, the determiner 20 produces an output indicative of whether the component light beam 1818 is considered to have been reflected from weed or other plant matter.

Accordingly, the determiner 20 produces a plurality of outputs 17, one for each component light beam 1818, which are then fed as inputs into the outcome determination system 16. In this example, each output comprises either a value ‘1’ indicating that the respective component light beam 1818 was reflected from a weed, or a value ‘0’ indicating that the respective component light beam 1818 was not reflected from a weed.

Turning now to the spatial analysis system 14, the image capturing device 22 may be a camera, such as but not limited to a digital camera. The device 22 is positioned relative to the optical device 18 so as to capture an image of the same area of interest 1815 onto which the component light beams 1818 are directed. The image capturing device 22 then sends image data corresponding to the captured to the second outcome determiner 24.

In this example, the second outcome determiner 24 comprises an image processor 2402, a weed identification module 2404 and a data storage 2406. The image processor 2402 and weed identification 2404 are each configured to execute software program instructions stored in the data storage 2406 to carry out their respective functions.

The image processor 2402 receives the image data from the device 22 and divides the image data into image segments, each segment including a portion of an image of the area of interest 1815 at which a component light beam 1818 is directed. In this example, since the distance between component light beams is known, and the position of the image capturing device 22 with respect to components of the optical device 18 is fixed, the image processor 2402 may automatically divide the image data into segments corresponding to the locations at which the component light beams 1818 fall in the area of interest 1815. The image processor 2402 then sends a series of input data corresponding to image segments to the weed identification module 2404.

The weed identification module 2404 then makes a decision for each image segment as to whether plant matter captured in the image segment is to be identified as weed. In this example, the module 2404 is configured to make decisions based on artificial intelligence techniques, such as but not limited to artificial neural network techniques.

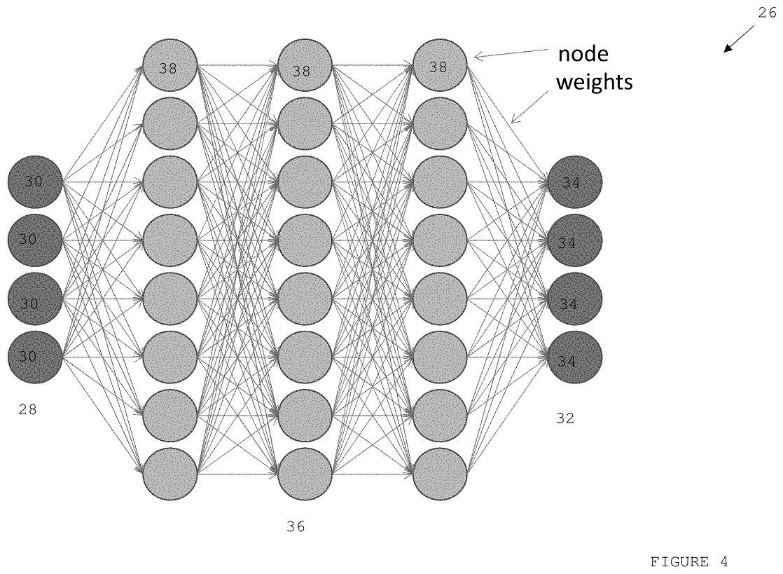

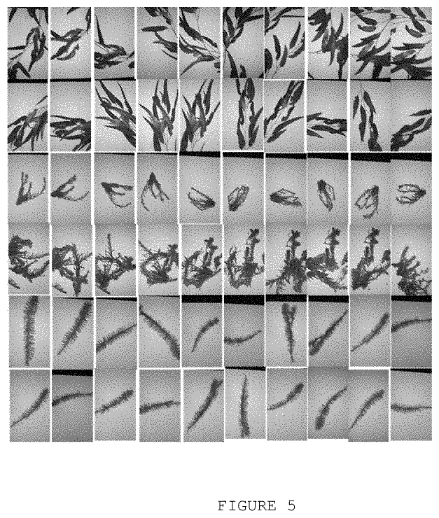

With reference to FIGS. 4 and 5, as will be familiar to a person skilled in the art, in an artificial neural network 26, there is typically an input layer 28 with multiple input nodes 30, an output layer 32 with multiple output nodes 34, and a hidden layer 36 of weighted nodes 38 providing a network of nodes interconnecting the input layer 28 and output layer 32, as shown in FIG. 4.

With reference to FIGS. 4 and 5, to produce meaningful outcomes, the neural network 26 is first trained using training data. This involves feeding each input node 30 with an input value, which (in reality) corresponds to a known set of desired outputs values. The neural network 26 assigns random weights to the weighted nodes 38. The neural network 26 will then calculate a set of output values for the output nodes 34 based on the inputs and random weights. These calculated output values will then be compared to the known set of desired outputs values, and the neural network 26 will systematically adjust the weights of the nodes 38 with a view to causing the next set of calculated output values to be closer to the desired output values.

In this example, the weed identification module 2404 is configured to implement a neural network. To train the weed identification module 2404, training data comprising hundreds of images of plant matter, including distorted, reflected and rotated versions of the same image, are fed as inputs (i.e. input nodes 30) into the weed identification module 2404. For illustrative purposes, examples of the training data images are provided in FIG. 5. Some of the images are images of weed, and some are images of other plant matter. It will be appreciated that the weed identification module 2404 comprises or interacts with any suitable image recognition software to detect an outline or shape of the images of the plant matter.

The weed identification module 2404 assigns random weights to weighted nodes 38, and output values (i.e. output nodes 34) are calculated. In this example, there is an output corresponding to each input. The calculated output values may be ‘0’ to indicate that the corresponding input is not an image of a weed, or a ‘1’ to indicate that the corresponding input is an image of a weed. Since in reality it is known which training data images actually correspond to weed and which do not, the desired outputs are known, which are then compared to the calculated output values. The weed identification module 2404 adjusts the values of the weighted nodes 38 and repeats the process to converge the calculated output to the desired output.

The weed identification module 2404 can be trained further with additional test data. Once the module 2404 is trained and the weightings of nodes are set, the system can be applied to real-world scenarios.

In operation, as the optical device 18 directs component light beams 1818 towards plant matter in the area of interest 1815, the image capturing device 22 will also capture an image of the area of interest 1815. The first and second outcome determiners 20, 24 may then operate concurrently to produce respective outputs 17, 19. Since each of the outputs 17, 19 are tied to component light beams 1818, each output produced by the first outcome determiner 20 can be paired with an output 19 from the second outcome determiner 24. Each pair of outputs 17, 19 is thus associated with a component beam 1818 and location in the area of interest 1815.

The outcome determination system 16 may cause an action or non-action to be taken at each location based on the values of each pair of outputs 17, 19. As mentioned above, the action may be to dispense a weed killer at a location if at least one output 17, 19 in the output pair corresponding to that location is a ‘1’.

As briefly described above, user operable controls, such as a dial, may be implemented in the system 10 to define the conditions for which an action is to be taken. For example, if a user would like to exercise caution when eradicating weed, the user may adjust the controls such that the weed killer is only dispensed at the respective location if the outputs 17, 19 for that location both return a ‘1’, i.e. a positive weed identification. Conversely, if a user would prefer to be more liberal with the dispensation of weed killer, the user can adjust the controls such that the weed killer is dispensed if at least one of the outputs 17, 19 for that location return a ‘1’.

It will be understood to persons skilled in the art of the invention that many modifications may be made without departing from the spirit and scope of the invention.

For example, other artificial intelligence techniques can be utilised instead of or in addition to neural network techniques, such as but not limited to Support Vector Machine (SVM) algorithms, and Normalised Difference Vegetation Indices (NDVIs).

Further, it will be appreciated that the first outcome determiner 20, second outcome determiner 24 and/or outcome determination system 16, may form part of the same computing device, and various functions of the first and second outcome determiners 20, 24 and outcome determination system 16 may be executed by the same processor. Alternatively, the first outcome determiner 20, second outcome determiner 24 and/or outcome determination system 16 may be embodied by separate devices.

As another example, rather than implementing first and second outcome determiners, the system 10 may instead communicate information from the optical device and image capturing device directly to the outcome determination system 16 for analysis. The outcome determination system 16 may be configured to analyse inputs from both the optical device and image capturing device together, implementing artificial intelligence techniques, such as a neural network, to produce an outcome.

It is to be understood that, if any prior art publication is referred to herein, such reference does not constitute an admission that the publication forms a part of the common general knowledge in the art, in Australia or any other country.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1-15. (canceled)

16. A detection system for detecting matter and distinguishing specific matter from other matter, the detection system comprising:
a) a dispenser arranged to dispense a substance to matter identified as specific matter upon instruction;
b) a spectral analysis system configured to at least assist in determining whether matter comprises the specific matter, the spectral analysis system comprising:
a light source capable of emitting a single light beam formed from a combination of at least three wavelengths or wavelength ranges appearing in sequence in the beam; and
an optical device including an optical element for directing the emitted light towards the matter, and a detector for detecting light reflected from the matter,
a first determiner that receives information indicative of the light reflected from the matter, determines intensities of each of the at least three wavelengths or wavelength ranges of the light reflected from the matter and determines a ratio of the intensities in order to produce an output indicative of whether the matter comprises the specific matter;
c) a spatial analysis system configured to detect a shape of the matter and at least assist in determining whether the matter comprises the specific matter based on the detected shape of the matter, the spatial analysis system comprising:
an image capturing device for capturing an image of the matter; and
a second determiner arranged to receive information indicative of the captured image from the image capturing device and produce an output, based on the detected shape of the matter, indicative of whether the matter comprises the specific matter;
d) an outcome determination system in communication with the dispenser, the outcome determination system arranged to receive the output from the spectral analysis system and the output from the spatial analysis system, and based on the outputs determine an outcome providing an ultimate indication of whether the matter is specific matter, and instruct the dispenser to dispense the substance on matter identified as specific matter if the outcome indicates the matter is specific matter;
wherein the outcome determination system further comprises a user operable selector capable of allowing a user to select a condition upon which the outcome depends thereby influencing when the dispenser dispenses the substance.

17. The detection system of claim 16, wherein the user operable selector is arranged to allow the user to select between the following conditions:
(i) at least one of the output from the spectral analysis system and the output from the spatial analysis system indicates that the matter is specific matter; and
(ii) each of the output from the spectral analysis system and the output from the spatial analysis system indicates that the matter is specific matter.

18. The detection system of claim 16, wherein the optical element is configured to receive the combined beam of light and direct a plurality of component light beams towards the matter.

19. The detection system of claim 16, wherein the optical element has first surface portions through which the plurality of component light beams is capable of being directed to the matter including the specific matter, the first surface portions having optical properties that are selected so that light intensity differences between the component light beams are reduced.

20. The detection system of claim 16, wherein the image capturing device is a camera.

21. The detection system of claim 16, wherein the matter is plant matter and the specific matter is unwanted plant matter.

22. The detection system of claim 21, wherein the dispenser is arranged to selectively dispense the substance onto the specific matter when the outcome determination system determines that the plant matter is unwanted plant matter.

23. The detection system of claim 16, wherein the spatial analysis system and the spectral analysis system performs respective analyses substantially simultaneously and/or in real-time.

24. The detection system of claim 16, wherein the outcome determination system is configured to determine whether the matter is specific matter using an artificial neural network.

25. A detection system for detecting matter and distinguishing specific matter from other matter, the detection system comprising:
e) a spectral analysis system configured to at least assist in determining whether matter comprises the specific matter, the spectral analysis system comprising:
a light source capable of emitting a single light beam formed from a combination of at least three wavelengths or wavelength ranges appearing in sequence in the beam; and
an optical device including an optical element for directing the emitted light towards the matter, and a detector for detecting light reflected from the matter,
a first determiner that receives information indicative of the light reflected from the matter, determines intensities of each of the at least three wavelengths or wavelength ranges of the light reflected from the matter and determines a ratio of the intensities in order to produce an output indicative of whether the matter comprises the specific matter;
f) a spatial analysis system configured to detect a shape of the matter and at least assist in determining whether the matter comprises the specific matter based on the detected shape of the matter, the spatial analysis system comprising:
an image capturing device for capturing an image of the matter; and
a second determiner arranged to receive information indicative of the captured image from the image capturing device and produce an output, based on the detected shape of the matter, indicative of whether the matter comprises the specific matter;
g) an outcome determination system arranged to receive the output from the spectral analysis system and the output from the spatial analysis system, and based on the outputs determine an outcome providing an ultimate indication of whether the matter is specific matter.
